(19) United States
(12) Patent Application Publication
(10) Pub. No.: US 2020/0081523 A1
(43) Pub. Date: Mar. 12, 2020

(54) SYSTEMS AND METHODS FOR DISPLAY

(71) Applicant: SHANGHAI UNITED IMAGING HEALTHCARE CO., LTD., Shanghai (CN)

(72) Inventor: Chang LIU, Shanghai (CN)

(73) Assignee: SHANGHAI UNITED IMAGING HEALTHCARE CO., LTD., Shanghai (CN)

(21) Appl. No.: 16/685,809

(22) Filed: Nov. 15, 2019

Related U.S. Application Data
(63) Continuation of application No. PCT/CN2017/084382, filed on May 15, 2017.

(51) Int. Cl. G06F 3/01; G06T 3/20; G06T 3/40; G16H 30/40

(52) U.S. Cl. CPC G06F 3/012; G06T 3/20; G16H 30/40; G06F 3/013; G06T 3/40

(57) ABSTRACT

The present disclosure discloses a display method. The method may include obtaining medical data and obtaining at least one of data related to a location of a user and data related to a focus of the user. The method may also include generating a virtual object based at least in part on the medical data. The virtual object may be associated with an application. The method may further include anchoring the virtual object to a physical location and managing the virtual object based on at least one of data related to the location of the user and data related to the focus of the user.

### CROSS-REFERENCE TO RELATED APPLICATIONS

This application is a continuation of International Application No. PCT/CN2017/084382 filed on May 15, 2017, the entire contents of which are hereby incorporated by reference.

### TECHNICAL FIELD

The present disclosure generally relates to the field of display, and in particular, to an interactive virtual reality system.

### BACKGROUND

In recent years, with the development of medical devices and visualization technologies, clinical diagnosis, medical research and other aspects have increasingly relied on medical imaging information. At present, a medical imaging system is largely implemented on a computer, displayed in a two-dimensional plane window, which is limited by a screen size and a resolution of the computer. A three-dimensional application is generally related to a three-dimensional rendering on a plane, which cannot provide an intuitive impression for a doctor. Therefore, it is desirable to provide an intuitive medical imaging system.

### SUMMARY

In an aspect of the present disclosure, a method is provided. The method may include obtaining medical data; obtaining at least one of data related to a location of a user and data related to a focus of the user; generating a virtual object based at least in part on the medical data, the virtual object being associated with an application; anchoring the virtual object to a physical location; managing the virtual object based on at least one of the data related to the location of the user and data related to the focus of the user.

In some embodiments, managing the virtual object based on at least one of the data related to the location of the user and the data related to the focus of the user may include: determining a relationship between the field of view of the user and the physical location based on at least one of data related to the location of the user and data related to the focus of the user; and managing the virtual object based on the relationship between the field of view of the user and the physical location.

In some embodiments, the relationship between the field of view of the user and the physical location may include: the field of view of the user includes the physical location. Managing the virtual object may include: displaying the virtual object at the physical location.

In some embodiments, the relationship between the field of view of the user and the physical location may include: the field of view of the user does not include the physical location. Managing the virtual object may include: displaying to the user a real scene within the field of view of the user.

In some embodiments, managing the virtual object may include at least one of displaying the application, zooming in the application, zooming out the application, and panning the application.

In some embodiments, generating the virtual object based at least in part on the medical data may include generating at least one of a mixed reality image, a virtual reality image, and an augmented reality image based at least in part on the medical data.

In some embodiments, obtaining the data related to the location of the user may include acquiring data related to a motion state of the user.

In some embodiments, obtaining data related to a motion state of the user may include obtaining data related to a motion state of a head of the user.

In some embodiments, the method may further include determining whether to display the virtual object based on the data related to a motion state of the head of the user.

In some embodiments, obtaining the data related to the focus of the user may include obtaining at least one of data related to a motion state of an eye of the user and imaging data of a corneal reflection of the user.

In another aspect of the present disclosure, a system is provided. The system may include a data acquisition module and a data processing module. The data acquisition module may be configured to obtain medical data; and obtain at least one of data related to a location of a user and data related to a focus of the user. The data processing module may be configured to generate a virtual object based at least in part on the medical data, the virtual object being associated with an application; anchor the virtual object to a physical location; and manage the virtual object based on at least one of data related to the location of the user and data related to the focus of the user.

In some embodiments, the data processing module may further be configured to determine a relationship between the field of view of the user and the physical location based on at least one of the data related to the location of the user and the data related to the focus of the user; and manage the virtual object based on the relationship between the field of view of the user and the physical location.

In some embodiments, the relationship between the field of view of the user and the physical location may include: the field of view of the user includes the physical location. Managing the virtual object may include displaying the virtual object at the physical location.

In some embodiments, the relationship between the field of view of the user and the physical location may include: the field of view of the user does not include the physical location. Managing the virtual object may include displaying to the user a real scene within the field of view of the user.

In some embodiments, the data processing module may further be configured to perform at least one of a display operation, a zoom in operation, a zoom out operation, and a pan operation on the application.

In some embodiments, the virtual object may include at least one of a mixed reality image, a virtual reality image, and an augmented reality image.

In some embodiments, the data related to the location of the user may include data related to the motion state of the user.

In some embodiments, the data related to the motion state of the user may include data related to a motion state of the head of the user.

In some embodiments, the data processing module may further be configured to determine whether to display the virtual object based on the data related to the motion state of the head of the user.

In some embodiments, the data related to the focus of the user may include at least one of data related to a motion state of an eye of the user and imaging data of a corneal reflection of the user. In some embodiments, the application may include at least one of a patient registration application, a patient management application, an image browsing application, and a printing application.

In some embodiments, the data acquiring module may include one or more sensors.

In some embodiments, the one or more sensors may include at least one of a scene sensor and an electrooculogram sensor.

In some embodiments, the medical data may be acquired by one or more of a positron emission tomography device, a computed tomography device, a magnetic resonance imaging device, a digital subtraction angiography device, an ultrasound scanning device, a thermal tomography device.

In another aspect of the present disclosure, a permanent computer readable medium storing a computer program is provided. The computer program may include instructions. The instructions may be configured to obtain medical data; obtain at least one of data related to a location of a user and data related to a focus of the user; generate a virtual object based at least in part on the medical data, the virtual object being associated with an application; anchor the virtual object to a physical location; and manage the virtual object based on at least one of the data related to a location of the user and the data related to the focus of the user.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities and combinations set forth in the detailed examples discussed below.

### BRIEF DESCRIPTION OF THE DRAWINGS

The drawings described herein are used to provide a further understanding of the present disclosure, all of which form a part of this specification. It should be expressly understood that the exemplary embodiment(s) of this disclosure are for the purpose of illustration and description only and are not intended to limit the scope of the present disclosure. In the drawings, like reference numerals represent similar structures.

FIGS. 1-A and 1-B are schematic diagrams illustrating an exemplary system according to some embodiments of the present disclosure;

FIG. 2 is a schematic diagram illustrating an exemplary computing device according to some embodiments of the present disclosure;

FIG. 3 is a schematic diagram illustrating exemplary hardware and/or software components of an exemplary mobile device of a terminal according to some embodiments of the present disclosure;

FIG. 4 is a schematic diagram illustrating an exemplary head-mounted display device according to some embodiments of the present disclosure;

FIG. 5 is a flowchart illustrating an exemplary process for displaying an image according to some embodiments of the present disclosure;

FIG. 6 is a block diagram illustrating an exemplary data acquisition module according to some embodiments of the present disclosure;

FIG. 7 is a block diagram illustrating an exemplary data processing module according to some embodiments of the present disclosure;

FIG. 8 is a flowchart illustrating an exemplary process for managing a virtual object according to some embodiments of the present disclosure;

FIG. 9 is a flowchart illustrating an exemplary process for managing a virtual object according to some embodiments of the present disclosure;

FIG. 10 is a flowchart illustrating an exemplary process for managing a virtual object according to some embodiments of the present disclosure;

FIG. 11 is a schematic diagram illustrating an exemplary application subunit according to some embodiments of the present disclosure;

FIG. 12 is a schematic diagram illustrating an exemplary application scenario of a head-mounted display device according to some embodiments of the present disclosure; and

FIG. 13 is a schematic diagram illustrating an exemplary application scenario of a head-mounted display device according to some embodiments of the present disclosure.

### DETAILED DESCRIPTION

In the following detailed description, numerous specific details are set forth by way of examples in order to provide a thorough understanding of the relevant disclosure. Obviously, drawings described below are only some examples or embodiments of the present disclosure. Those having ordinary skills in the art, without further creative efforts, may apply the present disclosure to other similar scenarios according to these drawings. It should be understood that these exemplary embodiments are only for the purpose of enabling those skilled in the relevant art to understand the present disclosure, and do not limit the scope of the present disclosure in any way. Unless apparent from the locale or otherwise stated, like reference numerals represent similar structures or operations throughout the several views of the drawings.

As used in the disclosure and the appended claims, the singular forms “a,” “an,” and “the” include plural referents unless the content clearly dictates otherwise. In general, the terms “comprise” and “include” merely prompt to include steps and elements that have been clearly identified, and these steps and elements do not constitute an exclusive listing. The methods or devices may also include other steps or elements.

The present disclosure includes some references to some modules in some the embodiments of the system in the present disclosure. However, a different number of modules can be used and run on the client and/or server. These modules are only used for illustration purposes, and different modules may be used in different aspects of the system and method.

The flowcharts used in the present disclosure illustrate operations that systems implement according to some embodiments of the present disclosure. It should be understood that the preceding or following operations may not be necessarily performed exactly in order. Instead, various steps may be processed in reverse sequence or simultaneously. Besides, one or more other operations may be added to the flow charts, or one or more operations may be omitted from the flow chart.

As used herein, the terms “comprising,” “may comprise,” “including,” or “may include” a feature (e.g., a numeral, a function, an operation, or a component such as a portion) represent the existence of a feature without excluding the existence of other features. As used herein, the terms “A or B,” “at least one of A and/or B” or “one or more in A and/or B” includes all possible combinations of A and B. For example, “A or B,” “at least one of A and B” or “at least one of A or B” may indicate all possible combinations below: (1) including at least one A, (2) including at least one B, or (3) including at least one A and at least one B.

As used herein, the term “configured (or set) to” may be used interchangeably according to the environment and terms “applicable to,” “capable,” “designed as,” “appropriately,” “manufactured as,” and “can”. The term “configured (or set) to” is not limited to “specifically designed in terms of hardware”. Moreover, the term “configured to” means that the device may perform operations along with other devices or components. For example, the term “processor is configured (or set) to perform A, B, and C” may refer to a general purpose processor (e.g., a central processor (CPU) or an application processor) that performs operations by one or more software programs stored in a storage device or a dedicated processor (e.g. embedded processor) for performing operations.

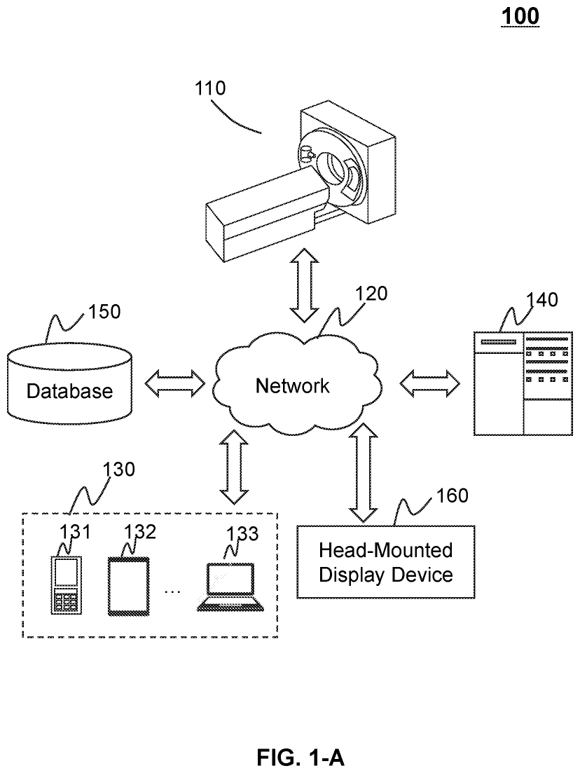

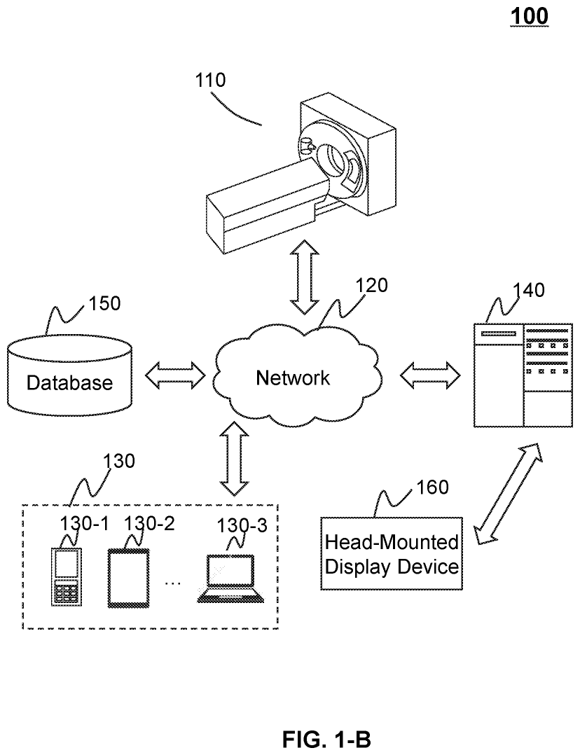

FIGS. 1-A and 1-B are schematic diagrams illustrating an exemplary display system 100 according to some embodiments of the present disclosure. The display system 100 may include a medical device 110, a network 120, a terminal 130, a data processing engine 140, a database 150, and a head-mounted display device 160. The one or more component of the display system 100 may communicate via the network 120. The display system 100 may include, but is not limited to, a virtual reality display system, an augmented reality display system, and/or a mixed reality display system.

The medical device 110 may collect data by scanning a target. The target of the scan may be an organ, a body, an object, an injured part, a tumor, or the like, or any combination thereof. For example, the target of the scan may be the head, the chest, the abdomen, an organ, the bone, blood vessel, or the like, or any combination thereof. As another example, the target of the scan may be one or more parts of vascular tissue, the liver, etc. The data collected by the medical device 110 may be image data. The image data may be two-dimensional image data and/or three-dimensional image data. In a two-dimensional image, the most subtle resolvable element may be a pixel. In a three-dimensional image, the most subtle resolvable element may be a voxel. In a three-dimensional image, the image may be composed of a series of two-dimensional slices or two-dimensional tomographic images. A point (or an element) in an image may be referred to as a voxel in a three-dimensional image, and may be referred to as a pixel in a two-dimensional tomographic image in which it is located. “Voxels” and/or “pixels” are merely for convenience of description and are not intended to limit the two-dimensional and/or three-dimensional images accordingly.

The medical device 110 may include, but is not limited to, a computed tomography (CT) device, a computed tomography angiography (CTA) device, a positron emission tomography (PET) device, a single photon emission computed tomography (SPECT) device, a magnetic resonance imaging (MRI) device, a digital subtraction angiography (DSA) device, an ultrasound scanning (US) device, a thermal tomography (TTM) device, etc.

The medical device 110 may be connected with the network 120, the data processing engine 140, and/or the head-mounted display device 160. In some embodiments, the medical device 110 may transmit data to the data processing engine 140 and/or the head-mounted display device 160. For example, the medical device 110 may send the collected data to the data processing engine 140 via the network 120. As another example, the medical device 110 may send the collected data to the head-mounted display device 160 via the network 120.

The network 120 may implement communications within the display system 100 and/or communications between the display system 100 and the outside of the display system 100. In some embodiments, the network 120 may implement a communication between the display system 100 and the outside of the display system 100. For example, the network 120 may receive external information, or send information to the outside of the display system 100. In some embodiments, the network 120 may implement a communication within the display system 100. Specifically, in some embodiments, the medical device 110, the terminal 130, the data processing engine 140, the database 150, the head-mounted display device 160, or the like, may access the network 120 via a wired communication, a wireless communication, or a combination thereof, and communicate via the network 120. As another example, the data processing engine 140 may obtain a user instruction from the terminal 130 via the network 120. As another example, the medical device 110 may transmit the collected data to the data processing engine 140 (or the head-mounted display device 160) via the network 120. As still another example, the head-mounted display device 160 may transmit data from the data processing engine 140 via the network 120.

The network 120 may include but is not limited to a local area network, a wide area network, a public network, a dedicated network, a wireless local area network, a virtual network, a metropolitan area network, a public switched telephone network, or the like, or any combination thereof. In some embodiments, the network 120 may include a plurality of network access points, such as wired or wireless access points, base stations, or network switching points, through which the data source may be connected to the network 120 to transmit information via the network 120.

The terminal 130 may receive, send, and/or display data or information. In some embodiments, the terminal 130 may include, but is not limited to, an input device, an output device, or the like, or any combination thereof. The input device may include, but is not limited to, a character input device (e.g., a keyboard), an optical reading device (e.g., an optical marker reader, an optical character reader), a graphic input device (e.g., a mouse, a joystick, a light pen), an image input device (e.g., a camera, a scanner, a fax machine), an analog input device (e.g., a language analog to digital conversion recognition system), or the like, or any combination thereof. The output device may include, but is not limited to, a display device, a printing device, a plotter, an image output system, a voice output system, a magnetic recording device, or the like, or any combination thereof. In some embodiments, the terminal 130 may be a device that has both input and output functions, such as a desktop computer, a laptop, a smartphone, a tablet computer, a personal digital assistant (PDA).

In some embodiments, the terminal 130 may include a mobile device 131 (or a mobile device 130-1), a tablet computer 132 (or a tablet computer 130-2), a laptop computer 133 (or a laptop computer 130-3), or the like, or any combination thereof. The mobile device may include a smart home device, a mobile phone, a personal digital assistant (PDA), a gaming device, a navigation device, a point of sale (POS) device, a laptop computer, a tablet computer, a film printer, a 3D printer, or the like, or any combination thereof. The smart home device may include a television, a digital multi-function disc (DVD) player, an audio player, a refrigerator, an air conditioner, a cleaner, an oven, a microwave oven, a washing machine, a dryer, an air purifier, a set-top box, a home automation control panel, a security control panel, a television set-top box, a game console, an electronic dictionary, an electronic key, a camcorder, an electronic photo frame, or the like, or any combination thereof.

The terminal 130 may be connected with the network 120, the data processing engine 140, and/or the head-mounted display device 160. In some embodiments, the terminal 130 may receive input information entered by a user and transmit the received information to the data processing engine 140 and/or the head-mounted display device 160. For example, the terminal 130 may receive data associated with an instruction entered by a user, and send the data associated with the instruction to the head-mounted display device 160 via the network 120. The head-mounted display device 160 may manage a display content based on the received data associated with the instruction.

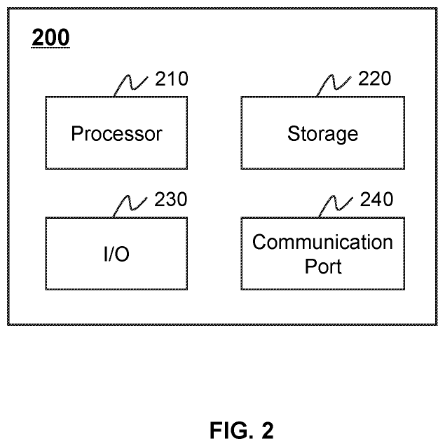

The data processing engine 140 may process data. The data may include image data, user input data, or the like. The image data may be two-dimensional image data, three-dimensional image data, or the like. The user input data may include a data processing parameter (e.g., a three-dimensional image reconstruction layer thickness, a layer spacing, a number of layers, etc.), an instruction associated with a system, or the like. The data may be data collected by the medical device 110, data read from the database 150, data obtained from the terminal 130 via the network 120, or the like. In some embodiments, the data processing engine 140 may be implemented on a computing device 200 having one or more components illustrated in FIG. 2.

The data processing engine 140 may be connected with the medical device 110, the network 120, the database 150, the terminal 130, and/or the head-mounted display device 160. In some embodiments, the data processing engine 140 may obtain data from the medical device 110 and/or the database 150. In some embodiments, the data processing engine 140 may send processed data to the database 150 and/or the head-mounted display device 160. For example, the data processing engine 140 may transmit processed data to the database 150 for storage or to the terminal 130. For example, the data processing engine 140 may process image data and transmit the processed image data to the head-mounted display device 160 for display. As another example, the data processing engine 140 may process user input data and transmit the processed user input data to the head-mounted display device 160. The head-mounted display device 160 may manage a display content based on the processed user input data.

The data processing engine 140 may include, but is not limited to, a central processing unit (CPU), an application specific integrated circuit (ASIC), an application specific instruction set processor (ASIP), a physical processing unit (PPU), a digital signal processor (DSP), a field-programmable gate array (FPGA), a programmable logic device (PLD), a processor, a microprocessor, a controller, a microcontroller, or the like, or any combination thereof.

It should be noted that the data processing engine 140 may be included in the display system 100, or may be implemented on a cloud computing platform to perform one or more corresponding functions. The cloud computing platform may include but is not limited to a data storing based storage cloud platform, a data processing based computing cloud platform, or an integrated cloud computing platform of data storing and data processing. The cloud platform used by the display system 100 may be a public cloud, a private cloud, a community cloud, a hybrid cloud, or the like, or any combination thereof. For example, according to an actual need, a medical image received by the display system 100 may be calculated and/or stored by the cloud platform, and a local processing module and/or the system simultaneously.

The database 150 may store data, instructions, and/or information, or the like. In some embodiments, the database 150 may store data obtained from the data processing engine 140 and/or the terminal 130. In some embodiments, the database 150 may store instructions, or the like, that the data processing engine 140 needs to execute.

In some embodiments, the database 150 may be connected to the network 120 to communication with one or more components (e.g., the medical device 110, the data processing engine 140, the head-mounted display device 160, etc.) of the display system 100. The one or more components of the display system 100 may obtain instructions or data stored in the database 150 via the network 120. In some embodiments, the database 150 may be directly connected to the one or more components of the display system 100. For example, the database 150 may be directly connected to the data processing engine 140. In some embodiments, the database 150 may be configured on one or more components of the display system 100 in the form of software or hardware. For example, the database 150 may be configured on the data processing engine 140.

The database 150 may be configured on a device that stores information using electrical energy, such as a memory, a random access memory (RAM), a read only memory (ROM), or the like. The RAM may include but not limited to a decatron, a selectron tube, a delay line memory, a Williams tube, a dynamic random access memory (DRAM), a static random access memory (SRAM), a thyristor random access memory (T-RAM), a zero capacitor random access memory (Z-RAM), or the like, or any combination thereof. The ROM may include but not limit to a bubble memory, a Twistor memory, a film memory, a plated wire memory, a magnetic core memory, a drum memory, an optical drive, a hard disk, a magnetic tape, an early non-volatile random access memory (NVRAM), a phase change memory, a magnetoresistive random access memory, a ferroelectric random access memory, a nonvolatile SRAM, a flash memory, an electronically erasable read only memory, an erasable programmable read only memory, a programmable read-only memory, a mask ROM, a floating-gate random access memory, a nano-RAM, a racetrack memory, a resistive random access memory, a programmable metallization cell, or the like, or any combination thereof. The database 150 may be configured on a device that stores information using magnetic energy, such as a hard disk, a floppy disk, a magnetic tape, a magnetic core memory, a bubble memory, a USB flash drive, a flash memory, or the like. The database 150 may be configured on a device that stores information optically, such as a CD, a DVD, or the like. The database 150 may be configured on a device that stores information in a magneto-optical manner, for example, a magneto-optical disk, or the like. The access mode of the information in the database 150 may be a random storage, a serial access storage, a read-only storage, or the like, or any combination thereof. The database 150 may be configured in a non-permanent memory, or a permanent memory. The storage device described above is merely an example, and a storage device that can be used in the display system 100 is not limited thereto.

The head-mounted display device 160 may obtain, transmit, and display an image. In some embodiments, the image may include a two-dimensional image and/or a three-dimensional image. In some embodiments, the image may include a mixed reality image, a virtual reality image, and/or an augmented reality image.

In some embodiments, the head-mounted display device 160 may obtain data from one or more of the medical device 110, the data processing engine 140, and/or the terminal 130. For example, the head-mounted display device 160 may obtain medical image data from the medical device 110. As another example, the head-mounted display device 160 may obtain an instruction entered by a user from the terminal 130. As still another example, the head-mounted display device 160 may obtain a stereoscopic image from the data processing engine 140 and display it. The head-mounted display device 160 may process data, display the processed data, and/or transmit the processed data to the terminal 130 for display. For example, the head-mounted display device 160 may process medical image data received from the medical device 110 to generate and display a stereoscopic medical image. As another example, the head-mounted display device 160 may transmit the generated stereoscopic image to the terminal 130 for display.

The head-mounted display device 160 may include a virtual reality device, an augmented reality display device, and/or a mixed reality device. For example, the head-mounted display device 160 may project a virtual image to provide a virtual reality experience for the user. As another example, the head-mounted display device 160 may project a virtual object while the user may view a real object through the head-mounted display device 160, to provide a mixed reality experience for the user. The virtual object may include a virtual text, a virtual image, a virtual video, or the like, or any combination thereof. As still another example, the mixed reality device may overlay a virtual image on a real image to provide a mixed reality experience for the user. The virtual image may include an image of a virtual object corresponding to a virtual space (e.g., a non-physical space). The virtual object may be generated based on computer processing. By way of example, the virtual object may include, but is not limited to, a two-dimensional (2D) image or movie object, a three-dimensional (3D) or four-dimensional (4D, i.e., a 3D object that change over time) image or movie object, or a combination thereof. For example, the virtual object may be an interface, a medical image (e.g., a PET image, a CT image, an MRI image), or the like. The real image may include an image of a real object corresponding to a real space (e.g., a physical space). For example, the real object may be a doctor, a patient, an operating table, or the like.

In some embodiments, the virtual reality device, the augmented reality display device, and/or the mixed reality device may include a virtual reality helmet, virtual reality glasses, a virtual reality eye mask, a mixed reality helmet, mixed reality glasses, a mixed reality eye mask, or the like, or any combination thereof. For example, the virtual reality device and/or the mixed reality device may include Google Glass™, Oculus Rift™, Hololens™, Gear VR™, or the like.

In some embodiments, the user may interact with a virtual object displayed on the head-mounted display device 160 via the head-mounted display device 160. The term “interaction” may include a physical interaction and a verbal interaction between a user and a virtual object. The physical interaction may refer to that the user performs a predefined gesture using his or her fingers, head, and/or other body parts that can be recognized by a mixed reality system as a request for the system to perform a predefined action. The predefined gesture may include, but is not limited to, pointing, grasping, and pushing a virtual object.

It should be noted that the above description of the display system 100 is merely provided for the purpose of illustration, and not intended to limit the scope of the present disclosure. For persons having ordinary skills in the art, modules may be combined in various ways, or connected with other modules as sub-systems. Various variations and modifications may be conducted under the teaching of the present disclosure. However, those variations and modifications may not depart the spirit and scope of this disclosure.

FIG. 2 is a schematic diagram illustrating an example computing device 200 according to some embodiments of the present disclosure. The data processing engine 140 may be implemented on the computing device 200. As shown in FIG. 2, the computing device 200 may include a processor 210, a storage 220, an input/output 230, and a communication port 240.

The processor 210 may execute computer instructions associated with the present disclosure or implement functions of the data processing engine 140. The computer instruction may be a program execution instruction, a program termination instruction, a program operation instruction, a program execution route, or the like. In some embodiments, the processor 210 may process image data obtained from the medical device 110, the terminal 130, the database 150, the head-mounted display device 160, and/or any other components of the display system 100. In some embodiments, the processor 210 may include one or more hardware processors, such as a microcontroller, a microprocessor, a reduced instruction set computer (RISC), an application specific integrated circuit (ASIC), a dedicated instruction set processor (ASIP), a central processor (CPU), a graphics processing unit (GPU), a physical processing unit (PPU), a microcontroller unit, a digital signal processor (DSP), a field programmable gate array (FPGA), an advanced RISC machine (ARM), a programmable logic device or any circuit or processor capable of performing one or more functions. The input/output 230 may input and/or output data, or the like. In some embodiments, the input/output 230 may enable a user to interact with the data processing engine 140. In some embodiments, the input/output 230 may include an input device and an output device. The input device may include a keyboard, a mouse, a touch screen, a microphone, or the like, or any combination thereof. Exemplary output devices may include a display device, a speaker, a printer, a projector, or the like, or any combination thereof. The display device may include a liquid crystal display, a light emitting diode based display, a flat panel display, a curved screen, a television device, a cathode ray tube, a touch screen, or the like, or any combination thereof.

The communication port 240 may be connected to the network 120 to facilitate data communication. The communication port 240 may establish a communication between the data processing engine 140, the medical device 110, the terminal 130, and/or the database 150. The communication may be a wired communication and/or a wireless communication. The wired communication may include, for example, a cable, a fiber optic cable, a telephone line, or the like, or any combination thereof. The wireless communication may include, for example, a Bluetooth communication, a wireless network communication, a WLAN link, a ZigBee communication, a mobile network communication (e.g., 3G, 4G, 5G network, etc.), or the like, or any combination thereof. In some embodiments, the communication port 240 may be and/or include a standardized communication port, such as RS232, RS485, etc. In some embodiments, the communication port 240 may be a dedicated communication port. For example, the communication port 240 may be designed in accordance with medical digital imaging and communication protocols.

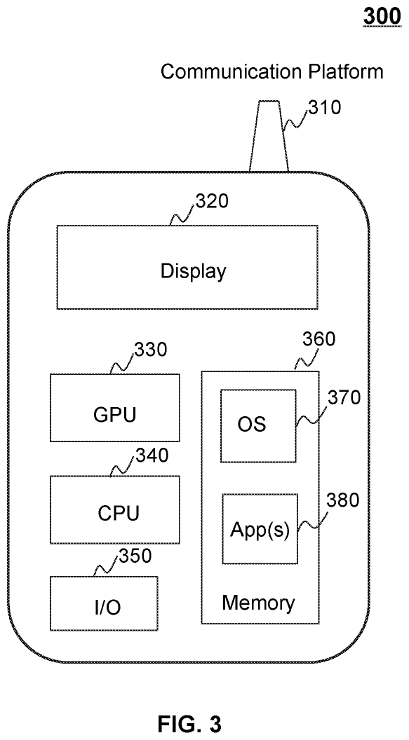

FIG. 3 is a schematic diagram illustrating exemplary hardware and/or software components of an exemplary mobile device 300 of the terminal 130 according to some embodiments of the present disclosure. As shown in FIG. 3, the mobile device 300 may include a communication platform 310, a display 320, a graphics processing unit 330, a central processing unit 340, an input/output 350, a memory 360, and a storage. In some embodiments, the mobile device 300 may include a bus or a controller. In some embodiments, a mobile operation system 370 and an application 380 may be loaded from the storage into the memory 360 and executed by the central processing unit 340. The application 380 may include a browser. In some embodiments, the application 380 may receive and display information relating to image processing or other information from the display system 100. User interactions with the information stream may be achieved via the input/output 350 and provided to other components of the display system 100, such as the data processing engine 140 and/or the head-mounted display device 160 via the network 120.

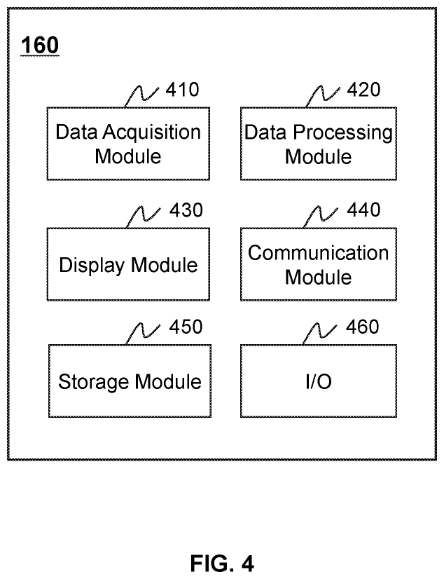

FIG. 4 is a schematic diagram illustrating an exemplary head-mounted display device 160 according to some embodiments of the present disclosure. As shown in FIG. 4, the head-mounted display device 160 may include a data acquisition module 410, a data processing module 420, a display module 430, a communication module 440, a storage module 450, and an input/output (I/O) 460.

The data acquisition module 410 may acquire data. The data may include medical data, data related to instructions, and/or scene data. The medical data may include data related to a patient. In some embodiments, the medical data may include data showing vital signs of the patient and/or transaction data of the patient. For example, the data showing the vital signs of the patient may include medical record data, prescription data, outpatient history data, physical examination data (e.g. a height, a weight, a body fat rate, a vision, a urine test data, a blood test data, etc.), a medical image (e.g., an X-ray image, a CT image, an MRI image, a RI image, an electrocardiogram), or the like, or any combination thereof. The transaction data of the patient may include patient admission information (e.g., outpatient data) and data related to an identity of the patient (e.g., specific ID number data for the patient set by a hospital, etc.). The data associated with instructions may include instructions and data that generates the instructions. In some embodiments, the data associated with instructions may include an instruction for managing the head-mounted display device 160. For example, the data associated with the instructions may include instructions entered by the user to manage the head-mounted display device 160. In some embodiments, the data associated with the instructions may include data that generates an instruction to manage the head-mounted display device 160. The data may include data related to a location of the user and/or data related to a focus of the user. The data related to the location of the user may include data related to a motion state of the user, for example, motion data of the head of the user, or the like. The data related to the focus of the user may include data that may be used to determine the focus of the user (e.g., motion data of an eye of the user and/or imaging data of a corneal reflection of the user). The scene data may include data required to construct a scene (e.g., a virtual reality scene, an augmented reality scene, and/or a mixed reality scene). As another example, the scene data may include data of a virtual object that constructs a virtual space (e.g., data required to draw a shape and a texture of a virtual object, such as data indicating a geometry, a color, a texture, a transparency, and other properties of the virtual object), data associated with a position and a direction of the virtual object, or the like.

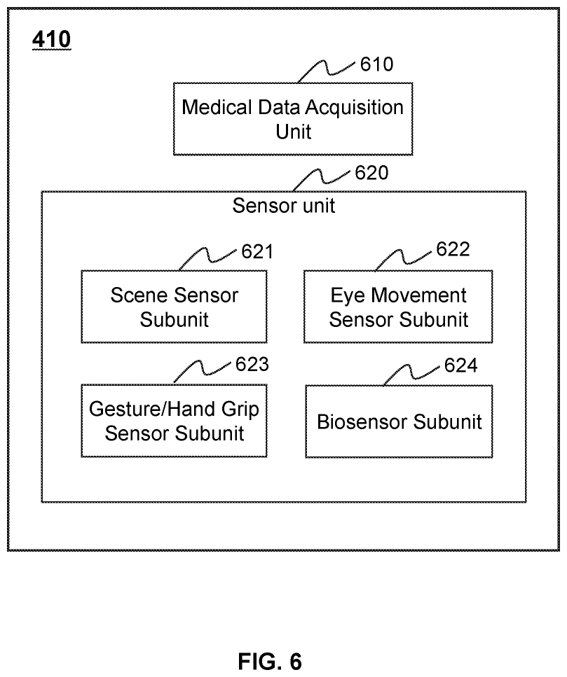

In some embodiments, the data acquisition module 410 may include one or more components shown in FIG. 6.

In some embodiments, the data acquisition module 410 may obtain data from one or more components (e.g., the medical device 110, the network 120, the data processing engine 140, the terminal, etc.) of the display system 100. For example, the data acquisition module 410 may obtain stereoscopic image data from the data processing engine 140. As another example, the data acquisition module 410 may obtain an instruction entered by the user via the terminal 130. In some embodiments, the data acquisition module 410 may collect data through a data collector. The data collector may include one or more sensors. The sensor may be an ultrasonic sensor, a temperature sensor, a humidity sensor, a gas sensor, a gas alarm, a pressure sensor, an acceleration sensor, an ultraviolet sensor, a magnetic sensor, a magnetoresistive sensor, an image sensor, a power sensor, a displacement sensor, or the like, or any combination thereof. In some embodiments, the data acquisition module 410 may transmit the obtained data to the data processing module 420 and/or the storage module 450.

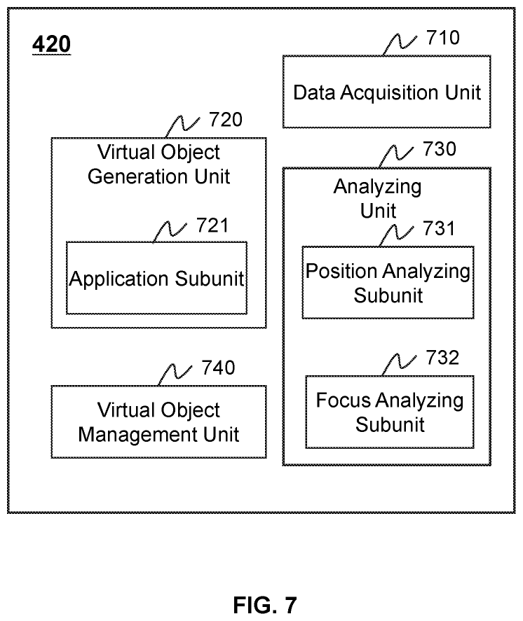

The data processing module 420 may process data. The data may include medical data and/or data related to instructions. In some embodiments, the data may be provided by the data acquisition module 410. In some embodiments, the data processing module 420 may include one or more components shown in FIG. 7.

The data processing module 420 may process medical data to generate a virtual object. In some embodiments, the virtual object may be associated with an application. For example, the data processing module 420 may process medical data of a patient (e.g., PET scan data of a patient) to generate a stereoscopic PET image. The PET image may be displayed by an image browsing application. In some embodiments, the data processing module 420 may insert the generated virtual object into a field of view of the user, such that the virtual object may expand and/or replace a real world view to provide a mixed reality experience for the user. In some embodiments, the data processing module 420 may anchor the generated virtual object to a physical location. The physical location may correspond to a location with a certain volume defined by a plurality of latitude coordinates, longitude coordinates, and altitude coordinates. For example, the physical location may be a wall of an operating room of a hospital, and the data processing module 420 may anchor a medical image browsing application to the wall.

The data processing module 420 may process data associated with an instruction to generate the instruction for controlling the head-mounted display device 160. The instruction for controlling the head-mounted display device 160 may include at least one of zooming in, zooming out, rotating, panning, and anchoring an image displayed on the head-mounted display device 160. The data processing module 420 may process at least one of data related to a location of the user and data related to a focus of the user to generate an instruction. In some embodiments, the data processing module 420 may process the data related to the location of the user to generate the instruction. For example, when the head of the user is turned to a physical location anchored with a virtual object, the data processing module 420 may control the head-mounted display device 160 to display the virtual object. When the head of the user is turned to a position other than the physical location, the data processing module 420 may control the head-mounted display device 160 not to display the virtual object. At this time, the user may view a real scene in the field of view through the head-mounted display device 160. As another example, when the user moves around in a virtual reality environment, the data processing module 420 may anchor the location of the virtual object, and the user may view the virtual reality object from different perspectives. When the user and the virtual object are stationary for a certain time period (e.g., 1 to 5 seconds), the data processing module 420 may relocate the virtual object for the user to view and/or interact with the virtual object. As still another example, when the user tilts his or her head at a certain oblique angle, the data processing module 420 may control the displayed virtual object to tilt at the same certain oblique angle in an oblique direction. As yet another example, when the user moves his or her head up, the data processing module 420 may zoom in on an upper portion of the virtual object. As yet another example, when the user moves his or her head down, the data processing module 420 may zoom in on a lower portion of the virtual object. As yet another example, when the user extends his or her head, the data processing module 420 may zoom in on the virtual object. When the user retracts his or her head, the data processing module 420 may zoom out on the virtual object. As yet another example, when the user turns his or her head counterclockwise, the data processing module 420 may control the head-mounted display device 160 to return to a previous menu. As still another example, when the user turns his or her head clockwise, the data processing module 420 may control the head-mounted display device 160 to display a content corresponding to a currently selected menu. In some embodiments, the data processing module 420 may process data related to a focus of the user, and generate an instruction to control the head-mounted display device 160. For example, when the focus of the user is focused on a virtual object for a predetermined time period (e.g., 3 seconds), the data processing module 420 may expand, enlarge, or the like, the virtual object.

In some embodiments, the data processing module 420 may include a processor to execute instructions stored in the storage module 450. The processor may be a standardized processor, a dedicated processor, a microprocessor, or the like. More descriptions of the processor may be found elsewhere in the present disclosure.

In some embodiments, the data processing module 420 may include one or more components shown in FIG. 7.

In some embodiments, the data processing module 420 may obtain data from the data acquisition module 410 and/or the storage module 450. For example, the data processing module 420 may obtain the medical data (e.g., PET scan data, etc.), the data related to the location of the user (e.g., motion data of the head of the user), and/or the data related to the focus of the use (e.g., motion data of the eye of the user, etc.) from the data acquisition module 410. In some embodiments, the data processing module 420 may process the received data and transmit the processed data to one or more of the display module 430, the storage module 450, the communication module 440, and/or the I/O (input/output) 460. For example, the data processing module 420 may process the medical data (e.g., PET scan data) received from the data acquisition module 410, and transmit a generated stereoscopic PET image to the display module 430 for display. As another example, the data processing module 420 may transmit a generated stereoscopic image to the terminal 130 via the communication module 440 and/or the I/O 460 for display. As yet another example, the data processing module 420 may process data associated with instructions received from the data acquisition module 410, generate an instruction for controlling the head-mounted display device 160 according to the data associated with instructions, and transmit the instruction to the display module 430 to control a display of an image by the display module 430.

The display module 430 may display information. The information may include text information, image information, video information, icon information, symbol information, or the like, or any combination thereof.

The display module 430 may display a virtual image and/or a real image, and provide a virtual reality experience, an augmented reality experience, and/or a mixed reality experience for the user. In some embodiments, the display module 430 may be transparent to some extent, and the user may view a real scene in the field of view through the display module 430 (e.g., an actual direct view of a real object), and the display module 430 may display a virtual image to the user to provide the user with a mixed reality experience. Specifically, for example, the display module 430 may project a virtual image in the field of view of the user such that the virtual image may be appeared next to a real world object to provide the user with a mixed reality experience. The actual direct view of the real object may refer to viewing a real object directly with human eyes, rather than viewing an image representation created by the object. For example, viewing a room through the display module 430 may allow the user to obtain an actual direct view of the room. While viewing a video of the room on a television may not be an actual direct view of the room. In some embodiments, the user cannot see the actual direct view of the real object in the field of view through the display module 430, and the display module 430 may display a virtual image and/or a real image to the user to provide the user with a virtual reality experience, an augmented reality experience, and/or a mixed reality experience. Specifically, for example, the display module 430 may project only a virtual image in the field of view of the user to provide the user with a virtual reality experience. As another example, the display module 430 may simultaneously project a virtual image and a real image in the field of view of the user to provide a user with a mixed reality experience.

The display module 430 may include a display. The display may include a liquid crystal display (LCD), a light emitting diode (LED) display, an organic LED (OLED) display, a microelectromechanical system (MEMS) display, an electronic paper display, or the like, or any combination thereof.

The communication module 440 may implement communications between the head-mounted display device 160 and one or more other components (e.g., the medical device 110, the network 120, the data processing engine 140, the terminal 130, etc.) of the display system 100. For example, the head-mounted display device 160 may be connected to the network 120 via the communication module 440 and receive a signal from the network 120 or transmit a signal to the network 120. In some embodiments, the communication module 440 may communicate with one or more components of the display system 100 via a wireless communication. The wireless communication may be a WiFi, a Bluetooth, a near field communication (NFC), a radio frequency (RF), or the like, or any combination thereof. The wireless communication may use a long term evolution (LTE), a LTE-enhanced (LTE-A), a code division multiple access (CDMA), a wideband CDMA (WCDMA), a universal mobile telecommunications system (UMTS), a wireless broadband (WiBro), or a global mobile telecommunications system (GSM). The wired communication may include at least one of a USB, a high definition multimedia interface (HDMI), a recommendation standard 232 (RS-232), and a plain old telephone service (POTS) as a communication protocol.

The storage module 450 may store commands or data related to at least one component of the head-mounted display device 160. In some embodiments, the storage module 450 may be connected to the data acquisition module 410, and store data acquired by the data acquisition module 410 (e.g., medical data, data related to an instruction, etc.). In some embodiments, the storage module 450 may be connected to the data processing module 420, to store instructions, programs, or the like, executed by a data module. Specifically, for example, the storage module 450 may store applications, intermediate software, application programming interfaces (APIs), or the like, or any combination thereof.

The storage module 450 may include a storage. The storage may include an internal storage and an external storage. The internal storage may include a volatile memory (e.g., a dynamic random access memory (DRAM), a static RAM (SRAM), a synchronous DRAM (SDRAM), etc.), or a non-volatile memory (e.g., a one-time programmable read only memory (OTPROM), a programmable ROM (PROM), an erasable programmable ROM (EPROM), an electrically erasable programmable ROM (EEPROM), a mask ROM, a flash ROM, a flash memory (e.g. a NAND flash or a NOR flash), a hard drive, or a solid state drive (SSD)). The external storage may include a flash drive, such as a compact flash (CF) memory, a secure digital (SD) memory, a micro SD memory, a mini SD memory, or a memory stick memory (Memory Stick™ memory card). The external storage may be connected to the head-mounted display device 160 via various types of interfaces functionally and/or physically.

The I/O (input/output) 460 may act as an interface, which may enable the head-mounted display device 160 to interact with a user and/or other devices. The other devices may include one or more components (e.g., the medical device 110) of the display system 100 and/or an external device. The external device may include an external computing device, an external storage device, or the like. More details regarding the external device may be found elsewhere in the present disclosure.

In some embodiments, the I/O 460 may include a USB interface, and for example, may further include an HDMI interface, an optical interface, or a D ultra-subminiature (D-sub) interface. Additionally or alternatively, the interface may include a mobile high definition connection (MHL) interface, a secure digital (SD) card/multimedia card (MMC) interface, or an infrared digital association (IrDA) standard interface. For example, the input/output interface may include one or more of a physical key, a physical button, a touch key, a joystick, a scroll wheel button, or a touch pad.

In some embodiments, the user may input information to the head-mounted display device 160 via the I/O 460. For example, the user may send an instruction to the head-mounted display device 160 via a joystick. In some embodiments, the head-mounted display device 160 may transmit data to or receive data from one or more components of the display system 100 via the I/O 460. For example, the I/O 460 may be a USB interface connected with the terminal 130. The head-mounted display device 160 may transmit a virtual image to the terminal 130 (e.g., a tablet computer) via the USB interface for display. In some embodiments, the head-mounted display device 160 may acquire data from an external device (e.g., an external storage device) via the I/O 460. For example, the I/O 460 may be a USB interface. A USB flash drive storing medical image data may transfer stored data (e.g., the medical image data) to the head-mounted display device 160 for processing and display.

It should be noted that the above description of the head-mounted display device 160 is merely provided for the purpose of illustration, and not intended to limit the scope of the present disclosure. For persons having ordinary skills in the art, after understanding the basic principles of the imaging apparatus, the modules may be combined in various ways, or connected with other modules as sub-systems without departing from the principles. According to some embodiments of the present disclosure, the head-mounted display device 160 may include at least one of the components described above, and may exclude one or more components or may include other accessories and components. According to some embodiments of the present disclosure, one or more components of the head-mounted display device 160 may be integrated into other devices (e.g., the terminal 130, etc.). The other devices may perform functions of the one or more components. As another example, the database 150 may be an independent component in communication with the data processing engine 140, or may be integrated into the data processing engine 140.

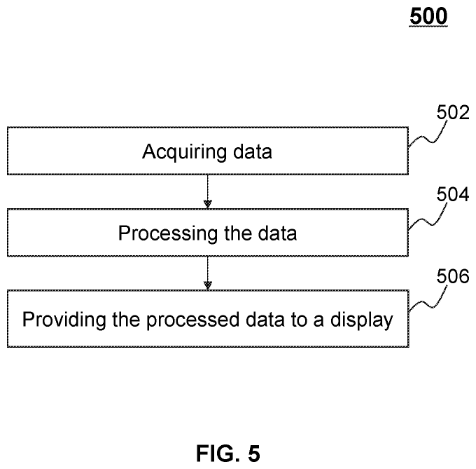

FIG. 5 is a flowchart illustrating an exemplary process for displaying an image according to some embodiments of the present disclosure. In some embodiments, the process 500 may be implemented by the head-mounted display device 160.

In operation 502, data may be acquired. The operation of acquiring data may be performed by the data acquisition module 410. The acquired data may include medical data, data related to a location of a user, and/or data related to a focus of the user as described in connection with the data acquisition module 410.

In operation 504, the data may be processed. The operation of processing data may be performed by the data processing module 420. The operation of processing data may include a combination of one or more of operations such as pre-processing, screening, and/or compensating the data. The data pre-processing may include denoising, filtering, dark current processing, geometric correction, or the like, or any combination thereof. For example, the data processing module 420 may perform a pre-processing operation on the acquired medical data. In some embodiments, the data processing module 420 may process the acquired medical data to generate a virtual image, as described in connection with the data processing module 420. In some embodiments, the data processing module 420 may manage a virtual object based on at least one of the data related to the location of the user, and the data related to the focus of the user.

In operation 506, the processed data may be provided to a display. In some embodiments, the display module 430 may display a virtual image. In some embodiments, the display module 430 may display a virtual image and a real image simultaneously.

It should be noted that the above description of the process for displaying an image is merely provided for the purpose of illustration, and not intended to limit the scope of the present disclosure. It should be understood that, for those skilled in the art, after understanding the principle of the system, it is possible to exchange or arbitrarily combine the various operations without deviating from this principle. Multiple variations and modifications may be made under the teachings of the present disclosure. For example, the acquired scan data may be stored for backup. Similarly, the operation for storing the backup may be added between any two operations in the flowchart.

FIG. 6 is a block diagram illustrating an example data acquisition module 410 according to some embodiments of the present disclosure. As shown in FIG. 6, the data acquisition module 410 may include a medical data acquisition unit 610 and a sensor unit 620.

The medical data acquisition unit 610 may obtain medical data. In some embodiments, the medical data obtained by the medical data acquisition unit 610 may include data showing vital signs of a patient, and/or transaction data of the patient. For example, the medical data acquisition unit 610 may acquire medical record data of the patient, prescription data, outpatient history data, medical examination data (e.g. a height, a weight, a body fat rate, a vision, a urine test data, a blood test data, etc.), a medical image (e.g., an X-ray image, a CT image, an MRI image, a RI image, an electrocardiogram, etc.), or the like, or any combination thereof. As another example, the medical data acquisition unit 610 may obtain patient admission data (e.g., outpatient data) and data related to an identity of the patient (e.g., specific ID number data for the patient set by the hospital, etc.). In some embodiments, the medical data acquisition unit 610 may obtain medical data from the medical device 110 and the data processing engine 140. For example, the medical data acquisition unit 610 may obtain a medical image (e.g., an X-ray image, a CT image, an MRI image, a RI image, an electrocardiogram, etc.) from the medical device 110. In some embodiments, the medical data acquisition unit 610 may transmit the acquired data to the data processing module 420 for processing, and/or to the storage module 450 for storage.

The sensor unit 620 may acquire a location of the user, a motion state of the user, a focus of the user, or the like, via one or more sensors. For example, the sensor unit 620 may measure a physical quantity or detect a position of a user by sensing at least one of a pressure recognition, a capacitance, or a dielectric constant change. As shown in FIG. 6, the sensor unit 620 may include a scene sensor subunit 621, an eye movement sensor subunit 622, a gesture/hand grip sensor subunit 623, and a biosensor subunit 624.

The scene sensor subunit 621 may determine a location and/or a motion state of the user in a scene. In some embodiments, the scene sensor subunit 621 may capture image data in a scene within its field of view, and determine the location and/or the motion state of the user based on the image data. For example, the scene sensor subunit 621 may be mounted on the head-mounted display device 160 to determine a change of the field of view of the user based on captured image data. The scene sensor subunit 621 may further determine the position and/or the motion state of the user in the scene. As another example, the scene sensor subunit 621 may be mounted outside the head-mounted display device 160 (e.g., mounted around a real environment of the user). The scene sensor subunit 621 may determine the position and/or the motion state of the user in the scene by capturing and analyzing the image data, tracking a posture and/or a movement of the user and a structure of the surrounding space.

The eye movement sensor subunit 622 may track and measure motion information of an eye of the user, track a movement of the eye of the user, and determine the field of view of the user and/or the focus of the user. For example, the eye movement sensor subunit 622 may acquire the motion information of the eye (e.g., an eyeball position, motion information of an eyeball, an eye gaze point, etc.) via one or more eye movement sensors, and track an eye movement. The eye movement sensor may track the field of view of the user by using at least one of an eye movement image sensor, an electrooculogram sensor, a coil system, a dual Purkinje system, a bright pupil system, and a dark pupil system. Additionally, the eye movement sensor subunit 622 may further include a miniature camera for tracking the field of view of the user. For example, the eye movement sensor subunit 622 may include an eye movement image sensor to determine the focus of the user by detecting imaging of corneal reflection of the user. The gesture/hand grip sensor subunit 623 may determine an input of the user by sensing a movement of a hand or a gesture of the user. For example, the gesture/hand grip sensor subunit 623 may determine whether the hand of the user is stationary, moving, or the like.

The biosensor subunit 624 may identify biological information associated with the user. For example, the biosensor may include an electronic nose sensor, an electromyogram (EMG) sensor, an electroencephalogram (EEG) sensor, an electrocardiogram (ECG) sensor, and an iris sensor.

It should be noted that the above description of the data acquisition module 410 is merely provided for the purpose of illustration, and not intended to limit the scope of the present disclosure. For persons having ordinary skills in the art, modules may be combined in various ways, or connected with other modules as sub-systems. Various variations and modifications may be conducted under the teaching of the present disclosure. However, those variations and modifications may not depart the spirit and scope of this disclosure. According to some embodiments of the present disclosure, the data acquisition module 410 may further include a magnetic sensor unit, etc.

FIG. 7 is a schematic diagram illustration an exemplary data processing module 420 according to some embodiments of the present disclosure. As shown in FIG. 7, the data processing module 420 may include a data acquisition unit 710, a virtual object generation unit 720, an analyzing unit 730, and a virtual object management unit 740. The virtual object generation unit 720 may include an application subunit 721. The analyzing unit 730 may include a position analyzing subunit 731 and a focus analyzing subunit 732.

The data acquisition unit 710 may acquire data that needs to be processed by the data processing module 420. In some embodiments, the data acquisition unit 710 may obtain data from the data acquisition module 410. In some embodiments, the data acquisition unit 710 may obtain medical data. For example, the data acquisition unit 710 may acquire a PET scan image of a patient. The image may be a two-dimensional image or a three-dimensional image. As another example, the data acquisition unit 710 may acquire transaction information of the patient. In some embodiments, the data acquisition unit 710 may acquire data related to a location of the user and/or data related to a focus of the user. For example, the data acquisition unit 710 may acquire a motion state of the head of the user and/or a motion state of an eye of the user. In some embodiments, the data acquisition unit 710 may transmit the acquired data to the virtual object generation unit 720 and/or the analyzing unit 730.

The virtual object generation unit 720 may generate a virtual object. In some embodiments, the virtual object generation unit 720 may obtain medical data from the data acquisition unit 710 and generate a virtual object based on the medical data. In some embodiments, the medical data may be provided by medical data acquisition unit 610. For example, the virtual object generation unit 720 may acquire a PET scan image of a patient and generate a corresponding virtual PET image based on the image. As another example, the virtual object generation unit 720 may acquire transaction information of the patient (e.g., an ID number of the patient) and generate a corresponding virtual object (e.g., an ID number of the patient in a virtual text form) based on the transaction information.

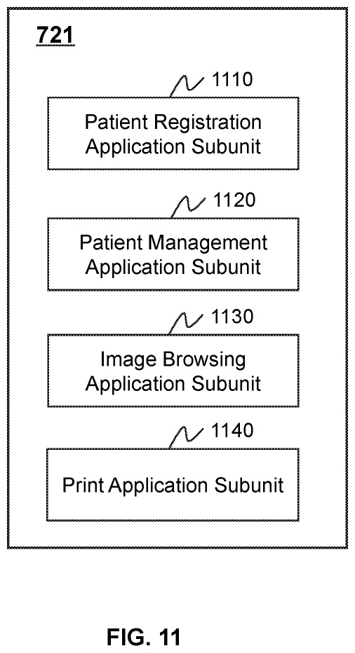

In some embodiments, the virtual object generation unit 720 may include an application subunit 721. The application subunit 721 may include an application. The application may implement various functions. In some embodiments, the application may include an application specified according to an external device (e.g., the medical device 110). In some embodiments, the application may include an application received from an external device (e.g., the terminal 130, the medical device 110, the data processing engine 140, etc.). In some embodiments, the application may include a preloaded application or a third party application downloaded from a server. Such as a dial-up application, a multimedia messaging service application, a browser application, a camera application, or the like. In some embodiments, the application may be generated in part based on the medical data. For example, the application may include an application for browsing patient information. The application may be generated in part based on the transaction information of the patient. As another example, the application may include a medical image browsing application. The application may be generated in part based on a medical scan image of the patient. In some embodiments, the application subunit 721 may include one or more components shown in FIG. 11.

The analyzing unit 730 may analyze data related to a location of the user and/or data related to a focus of the user. In some embodiments, the analyzing unit 730 may analyze at least one of the data related to the location of the user and the data related to the focus of the user, to obtain field of view information of the user. For example, the analyzing unit 730 may analyze head motion information, eye movement information, or the like, to obtain the field of view information of the user. In some embodiments, the analyzing unit 730 may analyze the data related to the focus of the user to obtain the focus information of the user. In some embodiments, the analyzing unit 730 may include a position analyzing subunit 731 and a focus analyzing subunit 732.

The position analyzing subunit 731 may analyze a position of the user and/or a position change of the user in a scene to obtain field of view information of the user. The location of the user in the scene may include a macroscopic position of an entire body of the user, or a location of a certain part of the body of the user (e.g., the head, a hand, an arm, a foot, etc.) in the scene. For example, the position analyzing subunit 731 may determine a location of the head of the user (e.g., an orientation of the head, etc.) to obtain the field of view information of the user. As another example, the position analyzing subunit 731 may determine a position change of the head of the user (e.g., a head orientation change, etc.) to obtain motion state information of the user.

The focus analyzing subunit 732 may determine a focus of the user. For example, the focus analyzing subunit 732 may determine the focus of the user based on eye movement information of the user. As another example, the focus analyzing subunit 732 may determine the focus of the user based on imaging of the corneal reflection of the user. In some embodiments, the focus analyzing subunit 732 may determine that the focus of the user remains on a virtual object for a predetermined time period. For example, the predetermined time may be in a range of 1-5 seconds. As another example, the predetermined time period may be greater than 5 seconds. In some embodiments, the focus analyzing subunit 732 may determine the field of view of the user based on the focus of the user. For example, the focus analyzing subunit may determine the field of view of the user based on the imaging of the corneal reflection of the user.

The virtual object management unit 740 may manage a virtual object. For example, the virtual object management unit 740 may zoom in, zoom out, anchor, rotate, and pan the virtual object. In some embodiments, the virtual object management unit 740 may acquire data from the analyzing unit 730 and manage the virtual object based on the acquired data.

In some embodiments, the virtual object management unit 740 may obtain field of view information of the user from the analyzing unit 730, and manage the virtual object based on the field of view information. For example, the virtual object management unit 740 may obtain information that the field of view of the user includes a physical location (e.g., a wall in an operating room) anchored with a virtual object (e.g., a CT image) from the position analyzing subunit 731 (or the focus analyzing subunit 732), and display the virtual object (e.g., a CT image) to the user. As another example, the virtual object management unit 740 may obtain information that the field of view of the user does not include a physical location (e.g., a wall in an operating room) anchored with a virtual object (e.g., a CT image) from the position analyzing subunit 731 (or the focus analyzing subunit 732), and may not display the virtual object e.g., a CT image) to the user. The user may view a real scene in the field of view through the head-mounted display device 160. In some embodiments, the virtual object management unit 740 may acquire focus data from the analyzing unit 730 and manage the virtual object based on the focus data. For example, the virtual object management unit 740 may obtain information that the focus of the user remains on a virtual object for a certain period of time (e.g., reaching or exceeding a time threshold) from the focus analyzing subunit 732, and generate an instruction to select and/or amplify the virtual object. In some embodiments, the virtual object management unit 740 may acquire the motion state information of the user from the analyzing unit 730, and manage the virtual object based on the motion state information.

It should be noted that the above description of the data processing module 420 is merely provided for the purpose of illustration, and not intended to limit the scope of the present disclosure. For persons having ordinary skills in the art, modules may be combined in various ways, or connected with other modules as sub-systems. Various variations and modifications may be conducted under the teaching of the present disclosure. However, those variations and modifications may not depart the spirit and scope of the present disclosure. According to some embodiments of the present disclosure, the data processing module 420 may include at least one of the components described above, and may exclude one or more components or may include other accessories and components. According to some embodiments of the present disclosure, the function of the data acquisition unit 710 may be integrated into the virtual object generation unit 720.

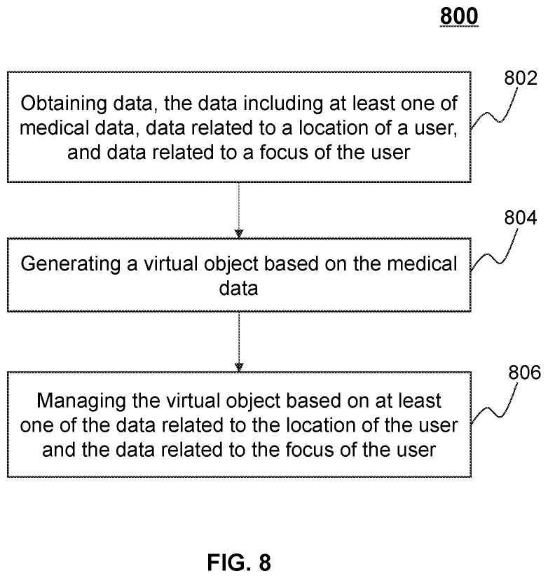

FIG. 8 is flowchart illustrating an exemplary process for managing a virtual object according to some embodiments of the present disclosure. In some embodiments, the process 800 may be implemented by the data processing module 420.

In operation 802, data may be acquired. The data may include at least one of medical data, data related to a location of the user, and data related to a focus of the user. In some embodiments, the operation of acquiring data may be performed by the data acquisition unit 710. For example, the data acquisition unit 710 may obtain a PET scan image of a patient. The image may be a two-dimensional image or a three-dimensional image. As another example, the data acquisition unit 710 may acquire transaction information of the patient.

In operation 804, a virtual object may be generated based on the medical data. In some embodiments, the operation of generating a virtual object may be performed by a virtual object generation unit 720. For example, the virtual object generation unit 720 may acquire a PET scan image of a patient and generate a corresponding virtual PET image based on the image. As another example, the virtual object generation unit 720 may acquire transaction information of the patient (e.g., an ID number of the patient), and generate a corresponding virtual object (e.g., an ID number of the patient in a virtual text form) based on the transaction information.

In operation 806, the virtual object may be managed based on at least one of data related to a location of the user and data related to a focus of the user. In some embodiments, the operation of managing the virtual object may be performed by the analyzing unit 730 and the virtual object management unit 740. For example, the analyzing unit 730 may determine the focus of the user based on data related to the focus of the user (e.g., imaging of a corneal reflection of the user). The virtual object management unit 740 may manage the virtual object based on the focus of the user. As another example, the analyzing unit 730 may obtain the field of view information of the user based on at least one of the data related to the location of the user and the data related to the focus of the user. The virtual object management unit 740 may manage the virtual object based on the field of view information of the user.

It should be noted that the above description of the process 800 for managing the virtual object is merely provided for the purpose of illustration, and not intended to limit the scope of the present disclosure. It should be understood that, for those skilled in the art, after understanding the principle of the system, it is possible to exchange or arbitrarily combine the various steps without deviating from this principle. Various variations and modifications may be conducted under the teaching of the present disclosure. However, those variations and modifications may not depart the spirit and scope of the present disclosure. For example, the acquired scan data may be stored for backup. Similarly, a backup operation may be added between any two operations in the flowchart.

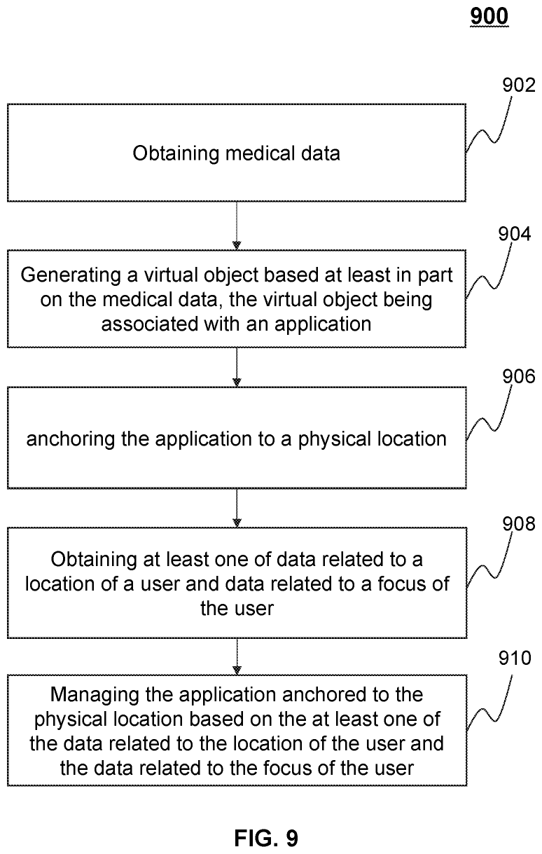

FIG. 9 is a flowchart illustrating an exemplary process for managing a virtual object according to some embodiments of the present disclosure. In some embodiments, the process 900 may be implemented by the data processing module 420.

In operation 902, medical data may be obtained. In some embodiments, the operation of acquiring data may be performed by the data acquisition unit 710. For example, the data acquisition unit 710 may obtain a PET scan image of a patient. The image may be a two-dimensional image or a three-dimensional image. As another example, the data acquisition unit 710 may acquire transaction information of the patient.

In operation 904, a virtual object may be generated based at least in part on the medical data. The virtual object may be associated with an application. The operation of generating the virtual object may be performed by the virtual object generation unit 720. In some embodiments, the application may be used to browse the virtual object. For example, the virtual object generation unit 720 may obtain a medical image of a patient. The medical image may be displayed by an image browsing application. In some embodiments, the virtual object may include the application. For example, the virtual object generation unit 720 may acquire the transaction information of the patient (e.g., an ID number of the patient) and generate an information management application of the patient (e.g., a patient registration application, a patient management application, etc.) based in part on the transaction information.

In operation 906, the application may be anchored to a physical location. The physical location may correspond to a location with a certain volume defined by a plurality of latitude coordinates, longitude coordinates, and altitude coordinates. The operation 906 may be performed by the virtual object generation unit 720. For example, the virtual object generation unit 720 may anchor a medical image browsing application to a wall of an operating room.

In operation 908, at least one of data related to a location of the user and data related to a focus of the user may be obtained. The operation may be performed by the data acquisition unit 710. For example, the data acquisition unit 710 may acquire data related to a motion state of the head of the user and/or a motion state of the eye of the user.

In operation 910, the application anchored to the physical location may be managed based on at least one of the data related to the location of the user and the data related to the focus of the user. The operation of managing the application may be performed by the analyzing unit 730 and the virtual object management unit 740. For example, when the user looks at the virtual object anchored to the physical location in the virtual world, the analyzing unit 730 may determine that the physical location is included in the field of view of the user. The virtual object management unit 740 may display the virtual object to the user at the physical location. When the user removes his or her eyes from the virtual object (e.g., the head of the user turns at a certain angle), the analyzing unit 730 may determine that the physical location is not included in the field of view of the user. The virtual object management unit 740 may stop (or cancel) the display of the virtual object. At this time, the user may view a real scene within the field of view.

It should be noted that the above description of the process 900 for managing the virtual object is merely provided for the purpose of illustration, and not intended to limit the scope of the present disclosure. It should be understood that, for those skilled in the art, after understanding the principle of the system, it is possible to exchange or arbitrarily combine the various steps without deviating from this principle. Various variations and modifications may be conducted under the teaching of the present disclosure. For example, the acquired scan data may be stored for backup. Similarly, a backup operation may be added between any two operations in the flowchart.

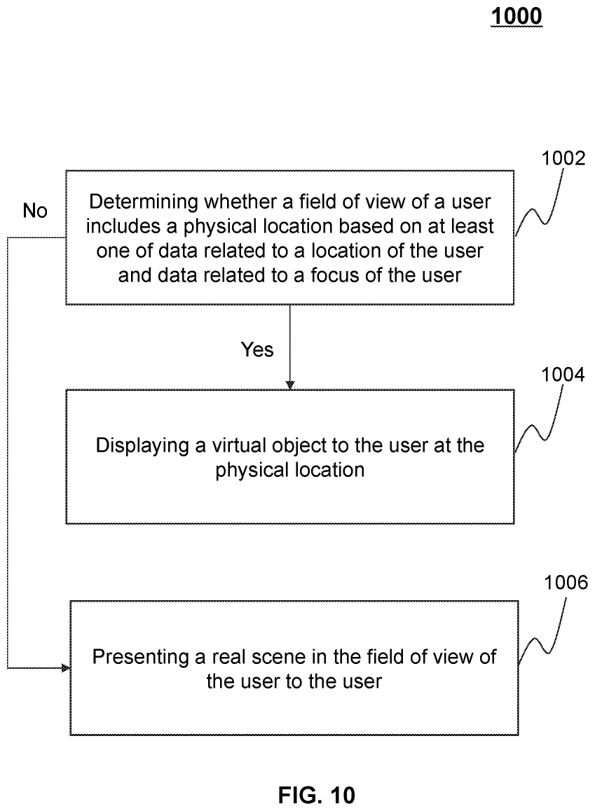

FIG. 10 is a flowchart illustrating an exemplary process for managing a virtual object according to some embodiments of the present disclosure. In some embodiments, the process 1000 may be implemented by the data processing module 420.

In operation 1002, a determination may be made as to whether the field of view of the user includes the physical location based on at least one of data related to a location of a user and data related to a focus of the user. In some embodiments, operation 1002 may be performed by the analyzing unit 730. In some embodiments, the analyzing unit 730 may determine whether the field of view of the user includes the physical location based on the data related to the location of the user. For example, the analyzing unit 730 may determine whether the user views a wall in an operating room based on head motion information of the user. In some embodiments, the analyzing unit 730 may determine whether the field of view of the user includes the physical location based on the data related to the focus of the user. For example, the analyzing unit 730 may determine whether the user views the wall in the operating room based on imaging of the corneal reflection of the user.

In response to a determination that the field of view of the user includes the physical location, in operation 1004, the virtual object may be displayed to the user at the physical location. In some embodiments, operation 1004 may be performed by the virtual object management unit 740. For example, if the user views the wall of the operating room, the virtual object management unit 740 may display the medical image browsing application to the user on the wall of the operating room. In response to a determination that the field of view of the user does not include the physical location, in operation 1006, a real scene may be presented in the field of view of the user. In some embodiments, operation 1006 may be performed by the virtual object management unit 740. For example, when the user looks at an operating table, at this time, the user cannot see the wall of the operating room, the virtual object management unit 740 may cancel the display of the medical image browsing application, and the user may view the real scene in the field of view, for example, a direct view of the operating table.

FIG. 11 is a schematic diagram illustrating an exemplary application subunit 721 according to some embodiments of the present disclosure. The application subunit 721 may include a patient registration application subunit 1110, a patient management application subunit 1120, an image browsing application subunit 1130, and a print application subunit 1140.

The patient registration application subunit 1110 may complete a registration of a patient. In some embodiments, the patient registration application subunit 1110 may manage transaction information of the patient. In some embodiments, the transaction information may be acquired by the data acquisition unit 710. For example, the data acquisition unit 710 may include an image sensor. The image sensor may acquire an image of an affected area of the patient and transmit the image to the patient registration application subunit 1110. As another example, the data acquisition unit 710 may obtain the transaction information from a patient system of a hospital, and transmit the information to the patient registration application subunit 1110.

The patient management application subunit 1120 may display examination information of a patient. The examination information of the patient may include medical examination data of the patient (e.g. a height, a weight, a body fat rate, a vision, urine test data, blood test data, etc.), a medical image (e.g., an X-ray image, a CT image, an MRI image, a RI image, an electrocardiogram, etc.), or the like, or any combination thereof. In some embodiments, the patient management application subunit 1120 may obtain the examination information of the patient from the database 150 and display the examination information. In some embodiments, the patient management application subunit 1120 may be displayed as a document folder, and may also be displayed on a virtual monitoring screen according to needs of the user, to imitate a computer interface operation familiar to the user.

The image browsing application subunit 1130 may browse an image. In some embodiments, the image browsing application subunit 1130 may display two-dimensional and/or three-dimensional information. For example, the image browsing application subunit 1130 may display a virtual object. In some embodiments, the image browsing application subunit 1130 may manage and determine a display form (e.g., an anchor display, a movement display) of the displayed content according to needs of the user. For example, the image browsing application subunit 1130 may manage the displayed virtual object according to an instruction sent from the virtual object management unit 740.

The print application subunit 1140 may print a related activity. In some embodiments, the print application subunit 1140 may complete an activity such as a film layout, an analog display of a film, and a virtual film preservation. In some embodiments, the print application subunit 1140 may communicate with a film printer or a 3D printer via the network 120 to complete film or 3D physical printing. In some embodiments, the print application may be displayed as a printer, to imitate a computer interface operation familiar to the user.

It should be noted that the above description of the application subunit 721 is merely provided for the purpose of illustration, and not intended to limit the scope of the present disclosure. For persons having ordinary skills in the art, modules may be combined in various ways, or connected with other modules as sub-systems. Various variations and modifications may be conducted under the teaching of the present disclosure. However, those variations and modifications may not depart the spirit and scope of the present disclosure. In some embodiments, the content displayed in the image browsing application may be displayed to a plurality of users as a common display item and an operation item of a plurality of mixed reality devices (or virtual reality devices), and the plurality of users may complete an interactive operation. For example, an operation performed based on virtual image information of the patient may be fed back to a plurality of users for discussion.

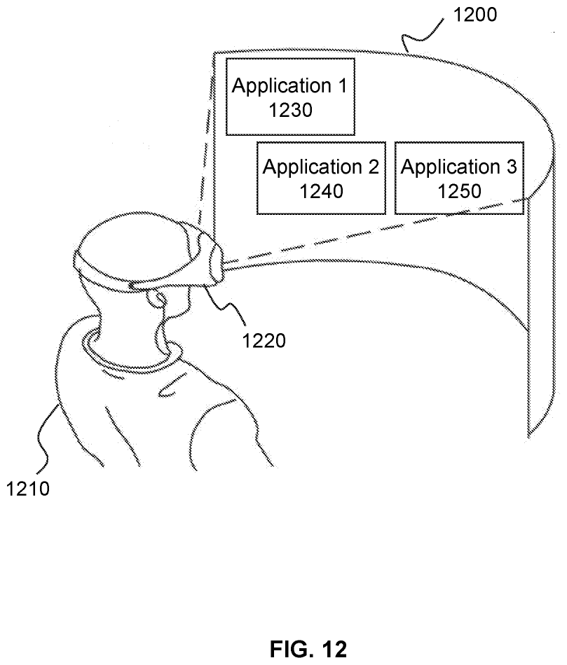

FIG. 12 is a schematic diagram illustrating an exemplary application scenario of the head-mounted display device 160 according to some embodiments of the present disclosure. As shown in FIG. 12, a user 1210 may wear a head-mounted display device 1220 to interact with one or more of an application 1230, an application 1240, and an application 1250 in a field of view 1200. The head-mounted display device 1220 may be a mixed reality device, an augmented reality device, and/or a virtual reality device.

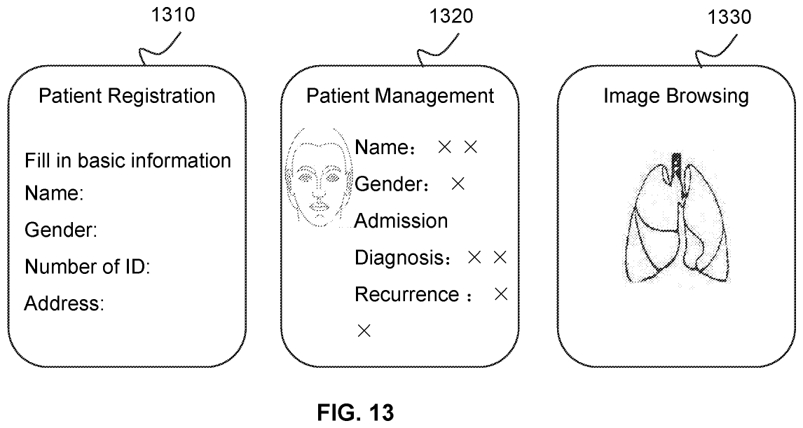

FIG. 13 is a schematic diagram illustrating an exemplary application according to some embodiments of the present disclosure. As shown in FIG. 13, an application may include a patient registration application 1310, a patient management application 1320, and an image browsing application function 1330. In some embodiments, a user may register patient information via the patient registration application 1310. In some embodiments, the user may view the patient information via the patient management application 1320. In some embodiments, the user may view a medical image of the patient (e.g., a PET image, a CT image, an MRI image, etc.) via the image browsing application 1330.

It should be noted that in the above, although “stop moving” may refer to that a user is standing or sitting completely still, as used herein, the term “stop moving” may include some degree of motion. For example, the user may be considered to be stationary if at least his/her feet are stationary, but one or more parts of a body of the user (e.g., the knee, the buttocks, the head, etc.) above the feet are moving. As used herein, “stop moving” may refer to a situation in which a user sits down but a leg, an upper body or the head of the user are moving. As used herein, “stop moving” may refer to that a user is moving, but after the user stops moving, the user is still in a range centered on the user with a relatively small diameter (e.g., 3 feet). In this example, the user may, for example, turn around within the range (e.g., to view a virtual object behind him/her), and the user may be considered as “not moving”. The term “not moving” may also refer to that a moving distance of the user in a predefined time period is less than a predetermined amount. As one of many examples, the user may be considered to be stationary if a moving distance of the user is less than 3 feet in any direction in a period of 5 seconds. As described above, this is only an example, and in other examples, both the movement amount and the period in which the movement amount is detected may change. When the head of a user is considered as stationary, it may indicate that the head of the user is stationary or the movement amount of the head is less than a threshold during a predetermined time period. For example, the head of the user may be considered to be stationary if the of the user pivots less than 45 degrees about an axis in a period of 5 seconds. Similarly, this is just an example and may change. When the movement of the user satisfies at least one of the movement conditions described above, the display system 100 may determine that the user is “not moving.”

Having thus described the basic concepts, it may be rather apparent to those skilled in the art after reading this detailed disclosure that the foregoing detailed disclosure is intended to be presented by way of example only and is not limiting. Various alterations, improvements and modifications to the present disclosure may occur and are intended to those skilled in the art, though not explicitly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various parts of this specification are not necessarily all referring to the same embodiment. In addition, certain features, structures, or characteristics may be combined as suitable in one or more embodiments of the present disclosure.

Moreover, it will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, various aspects of the present disclosure may be implemented entirely hardware, entirely software (including firmware, resident software, micro-code, etc.), or combining hardware and software. The above hardware or software may be referred to as “data block”, “module”, “engine”, “unit”, “component” or “system”. Furthermore, aspects of the present disclosure may take the form of a computer program product embodied in one or more computer readable media having computer readable program code embodied thereon.

A computer readable signal medium may include a propagated data signal with computer readable program code embodied therein, for example, in baseband or as part of a carrier wave. Such a propagated signal may take any of a variety of forms, including electro-magnetic, optical, or the like, or any suitable combination thereof. A computer readable signal medium may be any computer readable medium that is not a computer readable storage medium and that may communicated, or transport a program for use by or in connection with an instruction execution system, apparatus, or device. Program code embodied on a computer readable signal medium may be transmitted using any appropriate medium, including wireless, wireline, optical fiber cable, RF, or the like, or any suitable combination of the foregoing.

Computer program code for carrying out operations for aspects of the present disclosure may be written in any combination of one or more programming languages, including an object oriented programming language such as Java, Scala, Smalltalk, Eiffel, JADE, Emerald, C++, C #, VB. NET, Python, or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for Example, through the Internet using an Internet Service Provider) or in a cloud computing environment or offered as a service such as a Software as a Service (SaaS).

Furthermore, the recited order of processing elements or sequences, or the use of numbers, letters, or other designations therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” Unless otherwise stated, “about,” “approximate,” or “substantially” may indicate ±20% variation of the value it describes. Accordingly, in some embodiments, the numerical parameters set forth in the written description and attached claims are approximations that may vary depending upon the desired properties sought to be obtained by a particular embodiment. In some embodiments, the numerical parameters may take a prescribed effective digit into account and adopt a general method to approximate the numerical parameters. Notwithstanding that the numerical ranges and parameters setting forth the broad scope of some embodiments of the application are approximations, the numerical values set forth in the specific examples are reported as precisely as practicable.

For each of the patents, patent applications, patent application publications and other materials, such as articles, books, instructions, publications, documents, articles, etc., cited in this application are hereby incorporated by reference in their entirety. Application history documents that are inconsistent or conflicting with the contents of the present application are excluded, and documents (currently or later attached to the present application) that limit the widest range of the scope of the present application are also excluded. It should be noted that if the description, definition, and/or terms used in the appended application of the present disclosure is inconsistent or conflicting with the content described in the present disclosure, the use of the description, definition and/or terms of the Current disclosure shall prevail.

In closing, it is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

1-14. (canceled)

15. A method, comprising:
obtaining medical data;
obtaining at least one of data related to a location of a user and data related to a focus of the user;
generating a virtual object based at least in part on the medical data, the virtual object being associated with an application;
anchoring the virtual object to a physical location; and
managing the virtual object based on the at least one of the data related to the location of the user and the data related to the focus of the user.

16. The method of claim 15, wherein managing the virtual object based on the at least one of the data related to the location of the user and data related to the focus of the user comprises:
determining a relationship between the field of view of the user and the physical location based on the at least one of the data related to the location of the user and the data related to the focus of the user; and
managing the virtual object based on the relationship between the field of view of the user and the physical location.

17. The method of claim 16, wherein the relationship between the field of view of the user and the physical location includes: the field of view of the user includes the physical location, and managing the virtual object comprises:
displaying the virtual object at the physical location.

18. The method of claim 16, wherein the relationship between the field of view of the user and the physical location includes: the field of view of the user does not include the physical location, and managing the virtual object comprises:
displaying to the user a real scene in the field of view of the user.

19. The method of claim 15, wherein managing the virtual object comprises at least one of:
displaying the application, zooming in the application, zooming out the application, and panning the application.

20-24. (canceled)

25. A permanent computer readable medium storing a computer program, the computer program including instructions configured to:
obtain medical data;
obtain at least one of data related to a location of a user and data related to a focus of the user;
generate a virtual object based at least in part on the medical data, the virtual object being associated with an application;
anchor the virtual object to a physical location; and
manage the virtual object based on the at least one of the data related to the location of the user and the data related to the focus of the user.

26. A system, comprising:
at least one storage medium including a set of instructions;
at least one processor in communication with the at least one storage medium, wherein when executing the set of instructions, the at least one processor is configured to cause the system to:
obtain medical data;
obtain at least one of data related to a location of a user and data related to a focus of the user;
generate a virtual object based at least in part on the medical data, the virtual object being associated with an application;
anchor the virtual object to a physical location; and
manage the virtual object based on the at least one of the data related to the location of the user and the data related to the focus of the user.

27. The system of claim 26, wherein to manage the virtual object based on the at least one of the data related to the location of the user and the data related to the focus of the user, the at least one processor is configured to cause the system further to:
determine a relationship between a field of view of the user and the physical location based on at least one of the data related to the location of the user and the data related to the focus of the user; and
manage the virtual object based on the relationship between the field of view of the user and the physical location.

28. The system of claim 27, wherein the relationship between the field of view of the user and the physical location includes: the field of view of the user includes the physical location, and to manage the virtual object, the at least one processor is configured to cause the system further to:
display the virtual object at the physical location.

29. The system of claim 27, wherein the relationship between the field of view of the user and the physical location includes: the field of view of the user does not include the physical location, and to manage the virtual object, the at least one processor is configured to cause the system further to:
display to the user a real scene in the field of view of the user.

30. The system of claim 26, wherein the at least one processor is configured to cause the system further to:
perform at least one of a display operation, a zoom in operation, a zoom out operation, and a pan operation on the application.

31. The system of claim 26, wherein the virtual object includes at least one of a mixed reality image, a virtual reality image, and an augmented reality image.

32. The system of claim 26, wherein the data related to the location of the user includes data related to a motion state of the user.

33. The system of claim 32, wherein the data related to the motion state of the user includes data related to a motion state of a head of the user.

34. The system of claim 33, wherein the at least one processor is configured to cause the system further to:
determine whether to display the virtual object based on the data related to the motion state of the head of the user.

35. The system of claim 26, wherein the data related to the focus of the user includes at least one of data related to a motion state of an eye of the user and imaging data of a corneal reflection of the user.

36. The system of claim 26, wherein the application includes at least one of a patient registration application, a patient management application, an image browsing application, and a printing application.

37. The system of claim 26, further comprising:
one or more sensors.

38. The system of claim 37, wherein the one or more sensors includes at least one of a scene sensor and an electrooculogram sensor.

39. The system of claim 26, wherein the medical data is collected by at least one of a positron emission tomography device, a computed tomography device, a magnetic resonance imaging device, a digital subtraction angiography device, an ultrasonic scanning device, or a thermal tomography device.
